(19) United States
(12) Patent Application Publication
(10) Pub. No.: US 2023/0099958 A1
(43) Pub. Date: Mar. 30, 2023

(54) Locking Slide Clamp

(71) Applicants: Medical Components, Inc., Harleysville, PA (US); Innovative Medical Devices, LLC, Westport, CT (US)

(72) Inventors: Mark S. Fisher, Sellersville, PA (US); Kenneth M. Zinn, Westport, CT (US)

(21) Appl. No.: 17/953,664

(22) Filed: Sep. 27, 2022

Related U.S. Application Data
(60) Provisional application No. 63/249,857, filed on Sep. 29, 2021.

(51) Int. Cl. A61M 39/28

(52) U.S. Cl. CPC A61M 39/28

(57) ABSTRACT

A clamp includes a plate comprising an opening and a slit in communication with the opening; and a cap flexibly attached to the plate and comprising an engaging surface corresponding to the opening. A method for clamping a tube includes passing the tube through an opening in a plate of a clamp; sliding the tube aside towards a slit in the clamp, wherein the slit is in communication with the opening; and securing a cap in the opening so as to lock the clamp, preventing the tubing from entering the opening, wherein the cap is attached to the plate. The cap can flexibly adjust between an open position with the engaging surface outside the opening and a locked position with the engaging surface disposed within the opening. The clamp is suitable for use with medical tubing, such as hemodialysis catheters, intravenous lines, and peripherally inserted central catheters.

### CROSS-REFERENCE TO RELATED APPLICATION

This application claims the benefit of U.S. provisional application 63/249,857, filed on Sep. 29, 2021, the entire content of which is incorporated herein by reference.

### BACKGROUND OF THE INVENTION

### Field of the Invention

The subject matter disclosed herein generally relates to clamps. More specifically, the subject matter herein relates to a locking slide clamp, still more specifically a locking slide clamp adapted for clamping medical tubing.

### Background of the Related Art

Removable clamps are widely used to provide temporary clamping of medical tubing. Hemodialysis catheters, intravenous (IV) lines, and peripherally inserted central catheters (PICC) are examples of medical tubing that delivers fluid to and, in the case of hemodialysis catheters from, a patient. After insertion, the medical tubing remains in the patient over time, from hours to days to weeks to months to years. The medical tubing can include one or more tubes each having a lumen, that is an internal void space for carrying fluid. During the time the medical tubing is disposed in the patient, there is typically a need to start or stop the flow of fluid without removing the medical tubing from the patient. The control of fluid flow is accomplished with temporary or permanent equipment.

Temporary clamping is used variously in conjunction with operative permanent flow equipment, with medical tubing that does not include permanent flow equipment, or with inoperative permanent flow equipment. To provide permanent in place control of fluid flow, permanent on/off clamps or other permanent flow control equipment is incorporated with the medical tubing. However, the permanent flow control equipment can degrade or fail. Inserting, removing, changing, or repairing medical tubing can require a medical procedure. Temporary clamping allows continued operating of medical tubing to control the flow of fluid until a patient can access the procedure. A clamp applied to a lumen pinches the lumen at a pinch location impeding or preventing fluid flow at the pinch location.

As an example of medical tubing, percutaneous catheters are used for repetitively accessing the vascular system of a patient for hemodialysis. To place the percutaneous catheter in the body, the percutaneous catheter is inserted into a major vein, such as a femoral, subclavian or jugular vein. For long term maintenance dialysis, a jugular vein is generally the preferred insertion site. The catheter is percutaneous, with one end (i.e. proximal end) external to the body and the other end (i.e. distal end) dwelling in either the superior vena cava or the right atrium of the heart. The external portion of the catheter has connectors permitting attachment of blood lines leading to and from the hemodialysis machine. The hemodialysis catheter is left in the body after insertion, waiting to be used during an active dialysis session. Dialysis sessions occur frequently, for example every other day.

In order to minimize clotting within the hemodialysis catheter between dialysis sessions, the lumens of the hemodialysis catheter are typically filled with a diluted heparin solution (i.e., a “lock solution”) between the dialysis sessions. More particularly, after a dialysis session has been completed, a diluted heparin solution (i.e., the “lock solution”) is loaded into the lumens of the hemodialysis catheter. On/off clamps incorporated at the proximal end of the hemodialysis catheter (i.e., at the catheter's extracorporeal connectors) are closed to prevent the lock solution from draining out of the distal end of the catheter into systemic circulation. At the start of a hemodialysis session, the on/off clamps are opened and the lock solution is withdrawn from the hemodialysis catheter, whereupon the hemodialysis catheter is ready for use in a dialysis procedure.

Emergency hemodialysis repair kits are provided with hemodialysis catheters to allow a patient to go back and forth from dialysis and continue with dialysis when there is a problem with or failure of a value, or other part, until the patient is able to go in for a medical procedure for a more permanent fix. Emergency hemodialysis repair kits typically include one or more clamps.

Various temporary clamps are described in US utility patents U.S. Pat. Nos. 3,357,674, 4,248,401, 4,307,869, 4,434,963, 4,690,162, 4,932,629, 5,401,256, 5,593,392, 6,840,492, 7,124,996, 7,419,133, 8,276,875, 9,216,279, US design patent USD279314, and US9518667US utility patent application publications US2008/03129402A1, US2015/0224283A1, international patent application publication WO2002/089876A1, and Chinese patent application publication CN104138633A1.

Many temporary clamps are designed to facilitate placement and removal. However, such easy on, easy off temporary clamps can be susceptible to movement out of place. Movement out of place will cause the temporary clamp to fail to fully clamp, which can present health risk to the patient. However, solutions to reduced unwanted movement can increase the complexity of the clamp design.

The present invention is directed toward overcoming one or more of the above-identified problems.

### BRIEF SUMMARY OF THE INVENTION

In one or more embodiments, a cap is provided that can lock a clamp in place on one or more tubes, preventing unwanted movement of the one or more tubes out of the clamp.

In one or more embodiments, a clamp includes a plate comprising an opening and a slit in communication with the opening; and a cap flexibly attached to the plate and including an engaging surface corresponding to the opening. The slit is sized to receive a tubing and prevent fluid passage through the tubing when the tubing is placed in the slit. The plate comprises a first bounding edge defining the opening and a second bounding edge defining the slit. The first and second bounding edges are continuous. The first bounding edge comprises a receiving surface corresponding to the engaging surface. The cap can be secured in the opening, preventing the tubing from entering the opening.

In one or more embodiments, a method for clamping a tube includes passing the tube through an opening in a plate of a clamp; sliding the tube aside towards a slit in the clamp, where the slit is in communication with the opening; and securing a cap in the opening so as to lock the clamp, preventing the tubing from entering the opening, where the cap is attached to the plate.

In combination with one or more of the above embodiments, the opening may be adjacent the slit or at a distance from the slit. The receiving surface may have a profile that is flat or convex. The receiving surface may have a shape and a size about the same as a corresponding shape and size of the engaging surface.

In combination with one or more of the above embodiments, the cap may include a base portion at an end attached to the plate, a taper portion at an opposing end, and a plug portion between the base portion and the taper portion, wherein the plug portion includes the engaging surface. The cap may include a rib that can extend from the engaging surface. The rib can be adjacent the optional taper portion of the cap.

In combination with one or more of the above embodiments, the clamp may include a hinge between the cap and the plate, where the hinge attaches the cap to the plate. The plate, cap, and hinge may include a first polymeric material. The plate may include an insert comprising a second polymeric material. Alternately, the hinge may include a first polymeric material and the plate and cap may include a second polymeric material bonded to the first polymeric material.

In combination with one or more of the above embodiments, the plate may include a plurality of holes through the plate in a region of the plate around the opening and the cap can include a matching plurality of prongs.

In combination with one of more of the above embodiments, the clamp may include a reinforcing outer edge.

In combination with one or more of the above embodiments, the clamp may include an additional slit such that the clamp includes at least two slits. The clamp may include another additional slit such that the clamp includes at least three slits. The at least three slits may be arranged such that each is connected to the opening. Alternately, the plate may include a plurality of openings wherein each opening is connected to at least two of the at least three slits. When the clamp includes more than one slit and/or opening, each opening may be adjacent one of the slit and/or at a distance from one of the slits.

In combination with one or more of the above embodiments, the cap may include at least one non-clamping pass-through, wherein the non-clamping pass-through may be not aligned with any slit in the clamp.

While these potential advantages are made possible by technical solutions offered herein, they are not required to be achieved. The presently disclosed invention can be implemented to achieve technical advantages, whether or not these potential advantages, individually or in combination, are sought or achieved.

Further features, aspects, objects, advantages, and possible applications of the present invention will become apparent from a study of the exemplary embodiments and examples described below, in combination with the Figures, and the appended claims.

### BRIEF DESCRIPTION OF THE DRAWINGS

The above and other objects, aspects, features, advantages and possible applications of the present invention will be more apparent from the following more particular description thereof, presented in conjunction with the following drawings, in which:

FIG. 1A is a perspective view of a single slit clamp;

FIG. 1B is another perspective view of the clamp shown in FIG. 1A;

FIG. 1C is yet another perspective view of the clamp shown in FIG. 1A;

FIG. 1D is a close-up perspective view of the clamp shown in FIG. 1A;

FIG. 2A is a perspective view of a reinforced dual slit clamp;

FIG. 2B is another perspective view of the clamp shown in FIG. 2A;

FIG. 2C is yet another perspective view of the clamp shown in FIG. 2A;

FIG. 2D is a close-up perspective view of the clamp shown in FIG. 2A;

FIG. 3A is a perspective view of a strengthened reinforced dual slit clamp;

FIG. 3B is another perspective view of the clamp shown in FIG. 3A;

FIG. 3C is yet another perspective view of the clamp shown in FIG. 3A;

FIG. 3D is a close-up perspective view of the clamp shown in FIG. 3A;

FIG. 4 is perspective view of the clamp shown in FIG. 2A in a locked configuration; and

FIG. 5 is a perspective view of a dual slit clamp in an open configuration;

FIG. 6A is a perspective view of a dual slit clamp with pass-throughs;

FIG. 6B is another perspective view of the clamp shown in FIG. 6A;

FIG. 6C is a perspective view of the clamp shown in FIG. 6A in an unclamped configuration;

FIG. 6D is another perspective view of the clamp shown in FIG. 6C;

FIG. 7 is a cross-sectional view of a clamp with at least three slits; and

FIG. 8 is a perspective view of a hybrid clamp having a supporting insert.

### DETAILED DESCRIPTION OF THE INVENTION

The following description is of embodiments presently contemplated for carrying out the present invention. This description is not to be taken in a limiting sense, but is made merely for the purpose of describing the general principles and features of the present invention. The scope of the present invention should be determined with reference to the claims.

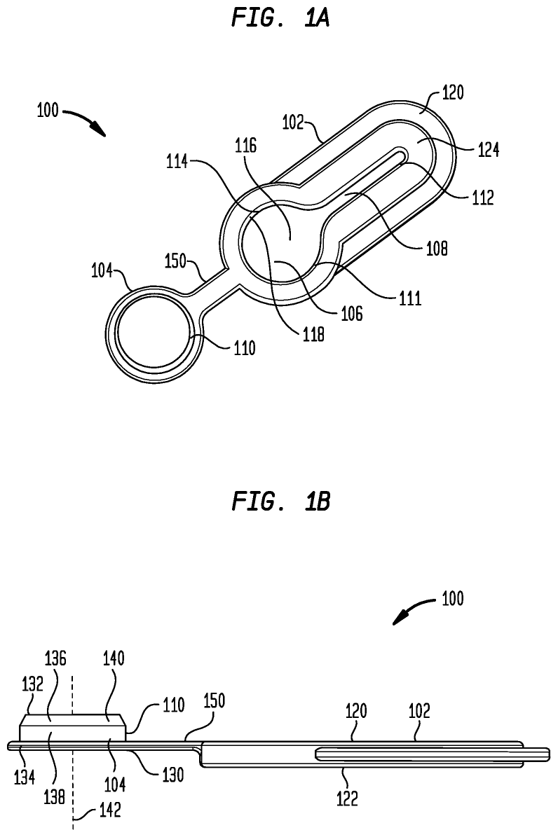

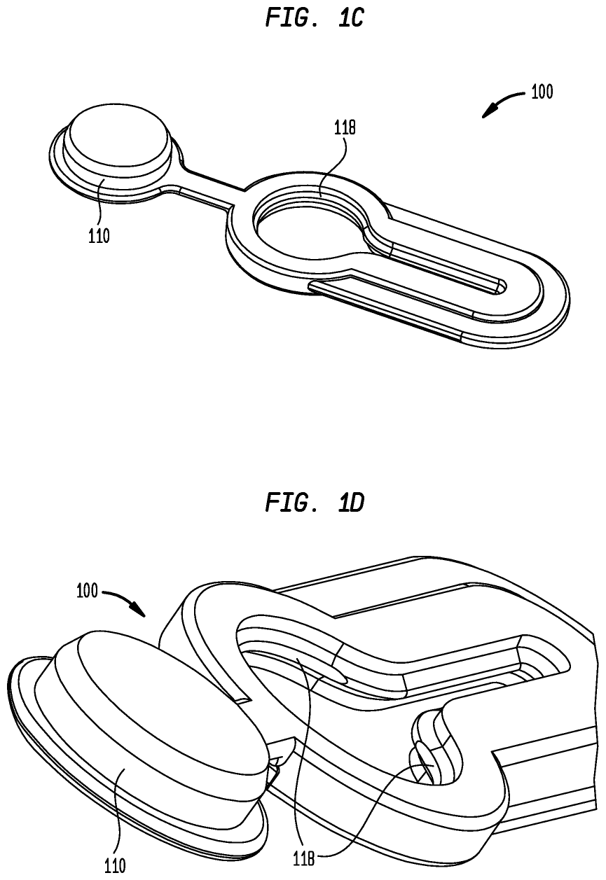

Referring to FIGS. 1A and 1B, clamp 100 includes plate 102 and cap 104 flexibly attached to plate 102. Plate 102 includes opening 106 and slit 108 in communication with opening 106. While maintaining communication between opening 106 and slit 108, opening 106 can be adjacent slit 108 as shown in FIG. 1A or at a distance from slit 108 (not shown). Cap 104 includes engaging surface 110 corresponding to opening 106.

Referring to FIG. 1A, plate 102 can include bounding edges 111, 112. Bounding edges 111, 112 are internal to plate 102. First bounding edge 111 defines opening 106 and second bounding edge 112 defines slit 108. Bounding edges 111, 112 are connected together. That is, bounding edges 111, 112 are part of a single interior bounding edge 114 defining a void 116 through plate 102 that includes opening 106 and slit 108. First bounding edge 111 can include receiving surface 118 corresponding to engaging surface 110. Referring to FIGS. 1C and 1D, receiving surface 118 has profile that is flat (as shown FIGS. 1A, 1B, 1C, 1D) or convex (not shown FIGS. 1A, 1B, 1C, 1D). Receiving surface 118 has shape and size about the same as corresponding shape and size of engaging surface 110. The shapes can be circular with respective receiving diameters defining the sizes or alternate shapes having respective average cross-sectional extents defining respective sizes. Referring again to FIG. 1A, when the corresponding sizes are about the same, the corresponding sizes are within design tolerances such that when cap 104 is disposed within opening 106, receiving surface 118 engages with engaging surface 110 with sufficient friction that cap 104 remains disposed within opening 106 without falling out, thus locking clamp 100.

Referring to FIG. 1B, plate 102 can include front side 120 on the front of plate 102 and back side 122 on the back of plate 102. Front denotes the side of plate 102 receiving cap 104. Referring again to FIG. 1A, side 120 can include ridge 124. Ridge 124 has the purpose of supporting the plate from deformation when receiving a tube into slit 108 and/or cap 104 into opening 106. Ridge 124 may extend around portions of bounding edge 111 and/or bounding edge 112. Thus, ridge 124 may extend around both the bounding edges 111, 112 (as shown in FIGS. 1A, 1B, 1C, 1D). Side 122 can likewise include a ridge (not shown.)

Referring again to FIG. 1B, cap 104 has end 130 attached to plate 102 and opposing end 132. Cap 104 can include base portion 134 at attached end 130, taper portion 136 at opposing end 132, and plug portion 138 between base portion 134 and taper portion 136. Base portion 134 extends from plug portion 138 such that base portion 134 has a larger cross-sectional size that plug portion 138. Base portion 134 provides a removal contact area to cap 104 so that a person can remove cap 104 from opening 106 to unlock clamp 100. Taper portion 136 includes taper surface 140 tapering from larger to smaller cross-sectional size along nominal axis 142 in the direction of opposing end 132.

Referring to FIGS. 1A, 1B, clamp 100 can include hinge 150 between plate 102 and cap 104. Hinge 150 attaches cap 104 to plate 102. The material of hinge 150 can be selected to be suitable for frequent bending to impart both flexibility and durability to the attachment between plate 102 and cap 104. The material of hinge 150, also termed herein the hinge material, can be a polymeric material. The material of other portions of clamp 100 can be the same as or different from the hinge material. Each portion of clamp 100 containing a single material can be integrally formed. For example, injection molding and 3D printing are suitable formation processes for polymeric materials. Hinge 150, cap 104, and plate 102 can contain the same material, that is a common material. When plate 102 is a single piece as shown in FIGS. 1A, 1B, the common material can be selected to be sufficiently hard for clamp 100 to hold its shape and sufficiently soft for clamp 100 to have both an open configuration and a closed configuration, that is for the clamp to be opened and closed. In the open configuration cap 104 is outside opening 106 and in the closed configuration cap 104 is engaged within opening 106. The common material can be a polymeric material. The polymeric material can be a thermoplastic, rubber, elastomer, and the like. For example, suitable materials include polypropylene, polyurethane, polyvinyl chloride, polyethylene coated polyvinyl chloride, silicone, and the like Suitable materials include conventional medical grade materials, for example medical grade polymeric materials. Alternately to a single material cap 104 and plate 102 can contain a different material to hinge 150. That is, hinge 150 may be form of a first material and cap 104 and plate 102 formed of a second material. The first material can be softer than the second material. The first material is selected to optimize hinge 150 flexing to open and close clamp 100. The second material is selected to optimize cap 104 and plate 102 holding their shape. The first and second materials can be a different polymeric materials from each other. Suitable polymeric materials include thermoplastic, rubber, elastomer, and the like. For example, suitable materials include polypropylene, polyurethane, polyvinyl chloride, polyethylene coated polyvinyl chloride, silicone, and the like Suitable materials for the first and second materials include conventional medical grade materials, for example medical grade polymeric materials. The different materials can be capable of being bonded. The different materials can both be polymeric. Thus, plate 102 and cap 104 can include a first polymeric material and the hinge 150 can include a second polymeric material bonded to the first polymeric material. A suitable combination is polypropylene for the first material and polyurethane for the second material.

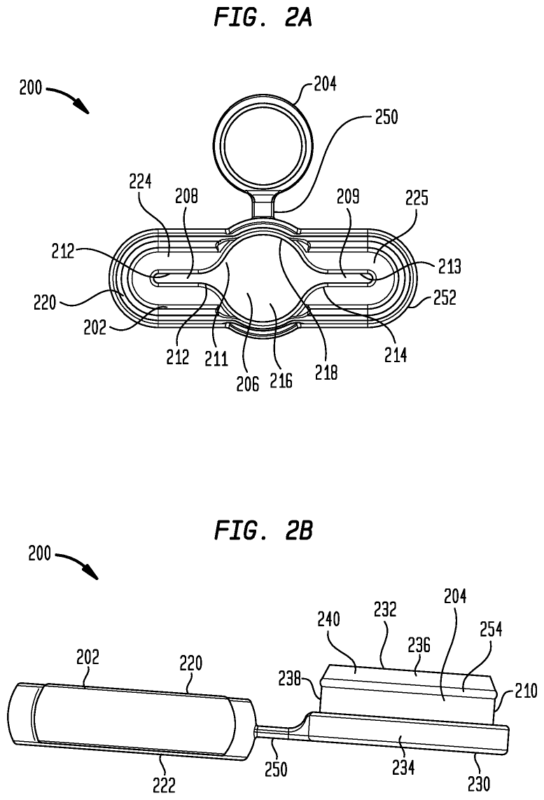

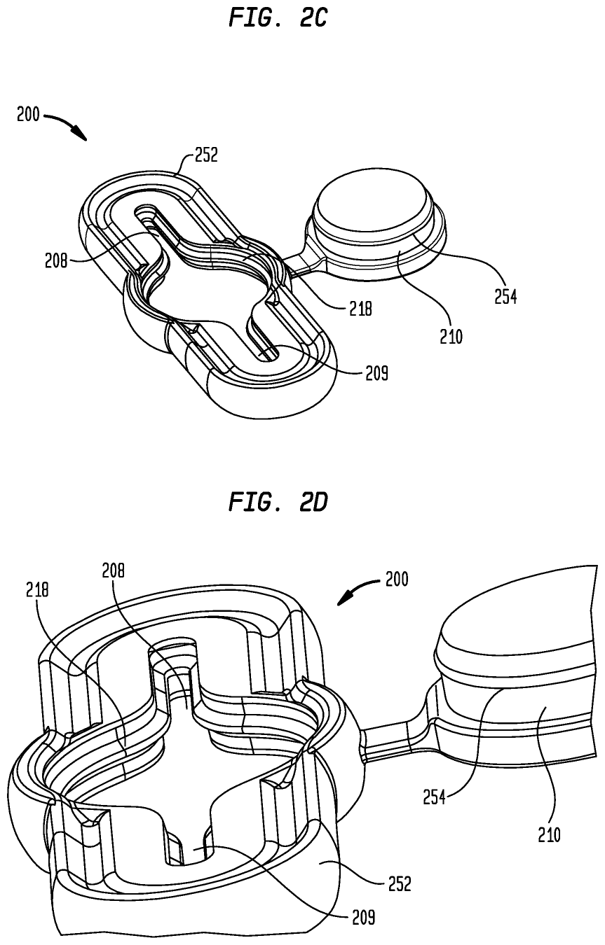

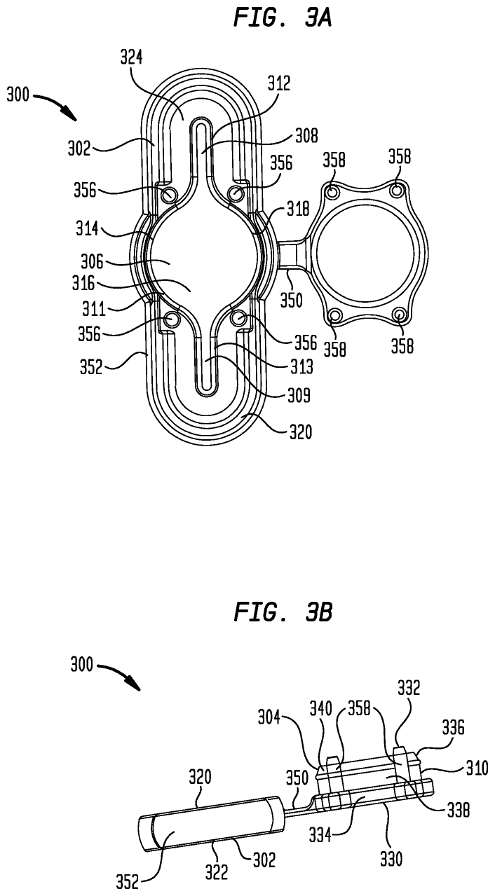

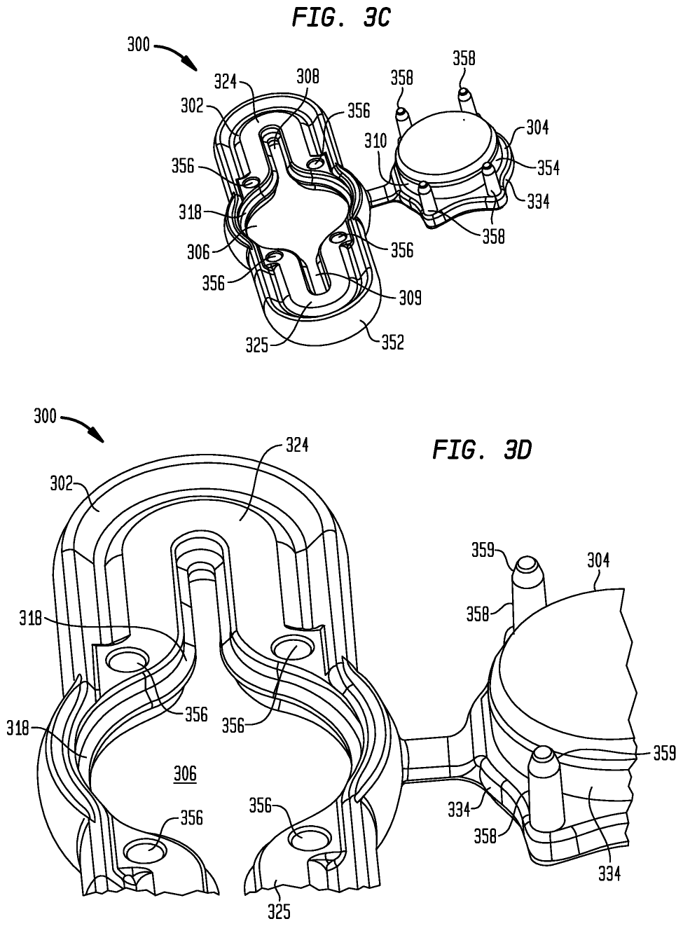

It will be understood that variations of the present clamp are contemplated, for example as shown in FIGS. 2A, 2B, 2C, 2D, 3A, 3B, 3C, or 3D. For example, referring to FIGS. 2C, and 2D, clamp 200 can include reinforcing outer edge 252, two slits 208, 209, and cap 204 that include rib 254 extending from engaging surface 210. Reinforcing outer edge 252 can extend along the perimeter of plate 202 surrounding plate 202. Rib 254 can be adjacent taper portion 246. Further, referring to FIGS. 3C and 3D, clamp 300 includes plate 302 that can further include a plurality of holes 356 therethrough in a region of plate 302 around opening 306 and clamp 300 includes cap 304 that can include a matching plurality of prongs 358. The matching plurality of prongs 358 corresponding to holes 356. Matching prongs 358 and holes 356 can strengthen the locking feature of clamp 300. Prongs 358 can extend from base portion 334 and include taper surfaces 359 at the ends opposite base portion 334. It will be understood that like numbers in FIGS. 2A, 2B, 2C, and 2D (e.g. 202, 204, 206, etc) and in 3A, 3B, 3C, and 3D (e.g. 302, 304, 306, etc.) to respective numbers in FIGS. 1A, 1B, 1C, and 1D (e.g. 102, 104, 106, etc) denote like parts (e.g. plate, cap, opening, etc).

Thus, referring to FIGS. 2A and 2B, clamp 200 includes plate 202 and cap 204 flexibly attached to plate 202. Cap 204 includes engaging surface 210 corresponding to opening 206. Plate 202 includes opening 206 and slits 208, 209 in communication with opening 206. While maintaining communication between opening 106 and slit 108, opening 106 can be adjacent each slit 208, 209 as shown in FIGS. 2A, 2B, 2C, 2D or at a distance from slit 208 and/or slit 209 (not shown). Clamp 200 includes reinforcing outer edge 252. Reinforcing outer edge 252 can extend along the perimeter of plate 202 surrounding plate 202.

Referring to FIG. 2A, plate 202 can include bounding edges 211, 212. Bounding edges 211, 212, 213 are internal to plate 202. First bounding edge 211 defines opening 206 and second bounding edge 212 defines slit 208 and third bounding edge 213 defines slit 209. Bounding edges 211, 212, 213 are connected together. That is, bounding edges 211, 212, 213 are part of a single interior bounding edge 214 defining a void 216 through plate 202 that includes opening 206 and slit 208. First bounding edge 211 can include receiving surface 218 corresponding to engaging surface 210. Referring to FIGS. 2C and 2D, receiving surface 218 has a profile that flat (not shown in FIGS. 2A, 2B, 2C, 2D) or convex (shown FIGS. 2A, 2B, 2C, 2D). Receiving surface 218 has a shape and size about the same as a corresponding shape and size of engaging surface 210. The shapes can be circular with respective receiving diameters defining the sizes or alternate shapes having respective average cross-sectional extents defining respective sizes. Referring again to FIG. 2A, when the corresponding sizes are about the same, the corresponding sizes are within design tolerances such that when cap 204 is disposed within opening 206, receiving surface 218 engages with engaging surface 210 with sufficient friction that cap 204 remains disposed within opening 206 without falling out, thus locking clamp 200.

Referring to FIG. 2B, plate 202 can include front side 220 on the front of plate 202 and back side 222 on the back of plate 202. Front denotes the side of plate 202 receiving cap 204. Referring again to FIG. 2A, side 220 can include ridges 224, 225 Ridges 224, 225 have the purpose of supporting the plate from deformation when receiving tubes into slits 208, 209 and/or cap 204 into opening 206. Ridge 222 may extend around portions of bounding edge 211 and/or bounding edges 212, 213. Thus, ridge 222 may extend around bounding edges 212, 213 and a part of bounding edge 211 as shown in FIGS. 2A, 2B, 2C, 2D. Side 222 can likewise include ridges (not shown.)

Referring again to FIG. 2B, cap 204 has end 230 attached to plate 202 and opposing end 232. Cap 204 can include base portion 234 at attached end 230, taper portion 236 at opposing end 232, and plug portion 238 between base portion 234 and taper portion 236. Base portion 234 extends from plug portion 238 such that base portion 234 has a larger cross-sectional size that base portion 224. Base portion 238 provides a removal contact area to cap 204 so that a person can remove cap 204 from opening 206 to unlock clamp 200. Taper portion 236 includes taper surface 240 tapering from larger to smaller cross-sectional size along the direction of a nominal axis that extends from attached end 230 to opposing end 232. Cap 204 can include rib 254 extending outwardly of engaging surface 210. Rib 254 can be adjacent taper portion 236.

Referring to FIGS. 2A and 2B, clamp 200 can include hinge 250 between plate 202 and cap 204. Hinge 250 attaches cap 204 to plate 202. The material of hinge 250 can be selected to be suitable for frequent bending to impart both flexibility and durability to the attachment between plate 202 and cap 204. The material of hinge 250, also termed herein the hinge material, can be a polymeric material. The material of other portions of clamp 200 can be the same as or different from the hinge material. Each portion of clamp 200 containing a single material can be integrally formed. For example, injection molding and 3D printing are suitable formation processes for polymeric materials. Hinge 250, cap 204, and plate 202 can contain the same material, that is a common material. When plate 202 is a single piece as shown in FIGS. 2A, 2B, the common material can be selected to be sufficiently hard for clamp 200 to hold its shape and sufficiently soft for clamp 200 to have both an open configuration and a closed configuration, that is for the clamp to be opened and closed. In the open configuration cap 204 is outside opening 206 and in the closed configuration cap 204 is engaged within opening 206. The common material can be a polymeric material. The polymeric material can be a thermoplastic, rubber, elastomer, and the like. For example, suitable materials include polypropylene, polyurethane, polyvinyl chloride, polyethylene coated polyvinyl chloride, silicone, and the like Suitable materials include conventional medical grade materials, for example medical grade polymeric materials. Alternately to a single material cap 204 and plate 202 can contain a different material to hinge 250. That is, hinge 250 may be form of a first material and cap 204 and plate 202 formed of a second material. The first material can be softer than the second material. The first material is selected to optimize hinge 250 flexing to open and close clamp 200. The second material is selected to optimize cap 204 and plate 202 holding their shape. The first and second materials can be a different polymeric materials from each other. Suitable polymeric materials include thermoplastic, rubber, elastomer, and the like. For example, suitable materials include polypropylene, polyurethane, polyvinyl chloride, polyethylene coated polyvinyl chloride, silicone, and the like. Suitable materials for the first and second materials include conventional medical grade materials, for example medical grade polymeric materials. The different materials can be capable of being bonded. The different materials can both be polymeric. Thus, plate 202 and cap 204 can include a first polymeric material and the hinge 250 can include a second polymeric material bonded to the first polymeric material. A suitable combination is polypropylene for the first material and polyurethane for the second material.

Further, referring to FIGS. 3A and 3B, clamp 300 includes plate 302 and cap 304 flexibly attached to plate 302. Referring to FIG. 3B, 304 includes engaging surface 310 corresponding to opening 306. Referring to FIG. 3A, 302 includes opening 306 and slits 308, 309 in communication with opening 306. While maintaining communication between opening 306 and slit 308, opening 306 can be adjacent each slit 308, 309 as shown in FIGS. 3A, 3B, 3C, 3D or at a distance from slit 308 and/or slit 309 (not shown). Clamp 300 includes reinforcing edge 352. Reinforcing outer edge 352 can extend along the perimeter of plate 302 surrounding plate 302.

Referring to FIG. 3A, plate 302 can include bounding edges 311, 312. Bounding edges 311, 312, 313 are internal to plate 302. First bounding edge 311 defines opening 306, second bounding edge 312 defines slit 308, and third bounding edge 313 defines slit 309. Bounding edges 311, 312, 313 are connected together. That is, bounding edges 311, 312, 313 are part of a single interior bounding edge 314 defining a void 316 through plate 302 that includes opening 306 and slit 308. Bounding edge 311 can include receiving surface 318 corresponding to engaging surface 310. Referring to FIGS. 3C and 3D, receiving surface 318 has profile that flat (not shown in FIGS. 3A, 3B, 3C, 3D) or convex (shown FIGS. 3A, 3B, 3C, 3D). Receiving surface 318 has a shape and size about the same as a corresponding shape and size of engaging surface 310. The shapes can be circular with respective receiving diameters defining the sizes or alternate shapes having respective average cross-sectional extents defining respective sizes.

Referring again to FIG. 3A, when the corresponding sizes are about the same, the corresponding sizes are within design tolerances such that when cap 304 is disposed within opening 306, receiving surface 318 engages with engaging surface 310 with sufficient friction that cap 304 remains disposed within opening 306 without falling out, thus locking clamp 300.

Referring to FIG. 3B, plate 302 can include front side 320 on the front of plate 302 and back side 322 on the back of plate 302. Front denotes the side of plate 302 receiving cap 304. Referring again to FIG. 3A, side 320 can include ridges 324, 325 Ridges 324, 325 have the purpose of supporting the plate from deformation when receiving tubes into slits 308, 309 and/or cap 304 into opening 306. Ridge 322 may extend around portions of bounding edge 311 and/or bounding edges 312, 313. Thus, ridge 322 may extend around bounding edges 312, 313 and a part of bounding edge 311 as shown in FIGS. 3A, 3B, 3C, 3D. Side 322 can likewise include ridges (not shown.)

Referring again to FIGS. 3A and 3B, plate 302 can include a plurality of holes 356 therethrough in a region of plate 302 around opening 306 and clamp 300 includes cap 304 that can include a matching plurality of prongs 358. The matching plurality of prongs 358 corresponding to holes 356. Matching prongs 358 and holes 356 can strengthen the locking feature of clamp 300. Prongs 358 can extend from base portion 334 and include taper surfaces 359 at the ends opposite base portion 334.

Referring again to FIG. 3B, cap 304 has end 330 attached to plate 302 and opposing end 332. Cap 304 can include base portion 334 at attached end 330, taper portion 336 at opposing end 332, and plug portion 338 between base portion 334 and taper portion 336. Base portion 334 extends from plug portion 338 such that base portion 334 has a larger cross-sectional size that base portion 324. Base portion 334 provides a removal contact area to cap 304 so that a person can remove cap 304 from opening 306 to unlock clamp 300. Taper portion 336 includes taper surface 340 tapering from larger to smaller cross-sectional size along the direction of a nominal axis that extends from attached end 330 to opposing end 332. Cap 304 can include rib 354 extending outwardly of engaging surface 310. Rib 354 can be adjacent taper portion 336.

Referring again to FIGS. 3A and 3B, clamp 300 can include hinge 350 between plate 302 and cap 304. Hinge 350 attaches cap 304 to plate 302. The material of hinge 350 can be selected to be suitable for frequent bending to impart both flexibility and durability to the attachment between plate 302 and cap 304. The material of hinge 350, also termed herein the hinge material, can be a polymeric material. The material of other portions of clamp 300 can be the same as or different from the hinge material. Each portion of clamp 300 containing a single material can be integrally formed. For example, injection molding and 3D printing are suitable formation processes for polymeric materials. Hinge 350, cap 304, and plate 302 can contain the same material, that is a common material. When plate 302 is a single piece as shown in FIGS. 1A, 1B, the common material can be selected to be sufficiently hard for clamp 300 to hold its shape and sufficiently soft for clamp 300 to have both an open configuration and a closed configuration, that is for the clamp to be opened and closed. In the open configuration cap 304 is outside opening 306 and in the closed configuration cap 304 is engaged within opening 306. The common material can be a polymeric material. The polymeric material can be a thermoplastic, rubber, elastomer, and the like. For example, suitable materials include polypropylene, polyurethane, polyvinyl chloride, polyethylene coated polyvinyl chloride, silicone, and the like Suitable materials include conventional medical grade materials, for example medical grade polymeric materials. Alternately to a single material cap 304 and plate 302 can contain a different material to hinge 350. That is, hinge 350 may be form of a first material and cap 304 and plate 302 formed of a second material. The first material can be softer than the second material. The first material is selected to optimize hinge 350 flexing to open and close clamp 300. The second material is selected to optimize cap 304 and plate 302 holding their shape. The first and second materials can be a different polymeric materials from each other. Suitable polymeric materials include thermoplastic, rubber, elastomer, and the like. For example, suitable materials include polypropylene, polyurethane, polyvinyl chloride, polyethylene coated polyvinyl chloride, silicone, and the like. Suitable materials for the first and second materials include conventional medical grade materials, for example medical grade polymeric materials. The different materials can be capable of being bonded. The different materials can both be polymeric. Thus, plate 302 and cap 304 can include a first polymeric material and the hinge 350 can include a second polymeric material bonded to the first polymeric material. A suitable combination is polypropylene for the first material and polyurethane for the second material.

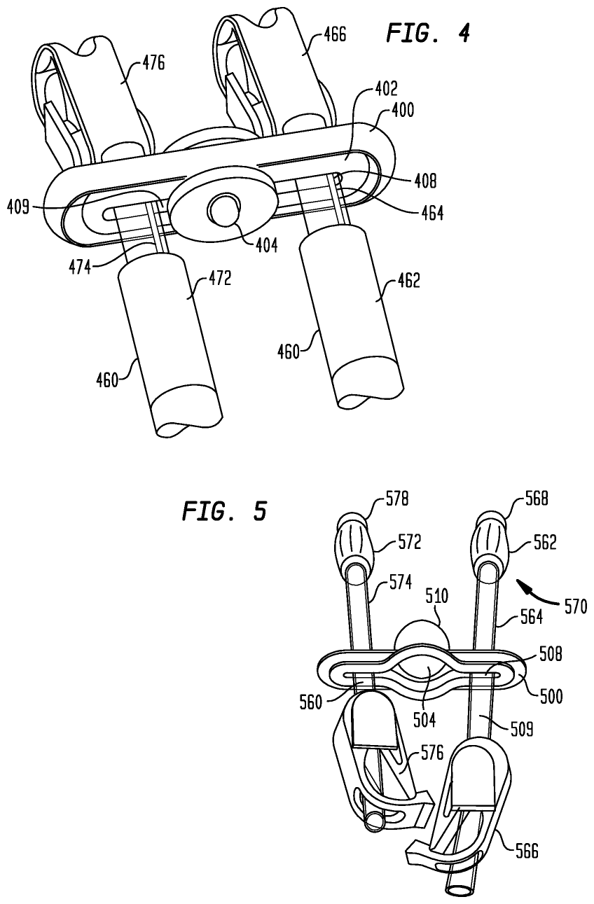

Referring to FIG. 4 and FIG. 5, clamps 400, 500 can be used with medical tubing 460, 560. Medical tubing 460 as shown in FIG. 4 is hemodialysis tubing. Medical tubing 460 includes connector 462 connectable to a suction line that delivers blood to a dialysis unit. Connector 462 includes tube 464, on/off clamp 466, and end piece (not shown). On/off clamp 466 and end piece can be red in color (not shown). Medical tubing 460 further includes connector 472 connectable to a return line that returns blood from a dialysis unit. Connector 472 includes tube 474, on/off clamp 476, and end piece (not shown). On/off clamp 476 and end piece can be blue in color (not shown). Each of on/off clamps 466, 476 can be a Halkey Roberts clamp. Slit 408 can have a width less than a cross-sectional size of tubes 464, 474. For example, slit 408 can have a width sufficiently smaller than the cross-section size of tubes 464, 474 that clamp 408 pinches tubes 464, 474 sufficiently to prevent flow of liquid through tubes 464, 474. It will be understood that although tubes 464, 474 can be cylindrical with circular cross-section other tube shapes are contemplated. A tube with circular cross-section has a size defined by an average cross-sectional diameter. When a tube does not have circular cross-section, for example a D cross-section, the size of the tube can be defined as the largest width across. When medical tubes 464, 474 are hemodialysis tubes, medical tubes 564, 474 can each have a size typical of hemodialysis lumens, such as from about 2 French (0.66 mm) to about 20 French (6.66 mm), for example from about 5 French (1.66) to about 17 French (5.66 mm), for example from about 7 French (2.33 mm) to about 15 French (5 mm). It will be understood that although hemodialysis catheters can include two connectors as shown in FIG. 4, alternately, hemodialysis catheters can include three connectors, for example when in addition to attaching to two hemodialysis lumens the catheter includes a third lumen for non-hemodialysis-related access. Clamp 400 can be included with an emergency repair kit for a hemodialysis catheter. It will be understood that clamp 400 can be used with peripherally inserted central catheter (PICC) tubing. The design of clamp 400 when used with PICC tubing is similar to the design of clamp 400 when used with hemodialysis tubing except the dimensions differ because PICCs are smaller in size than hemodialysis catheters.

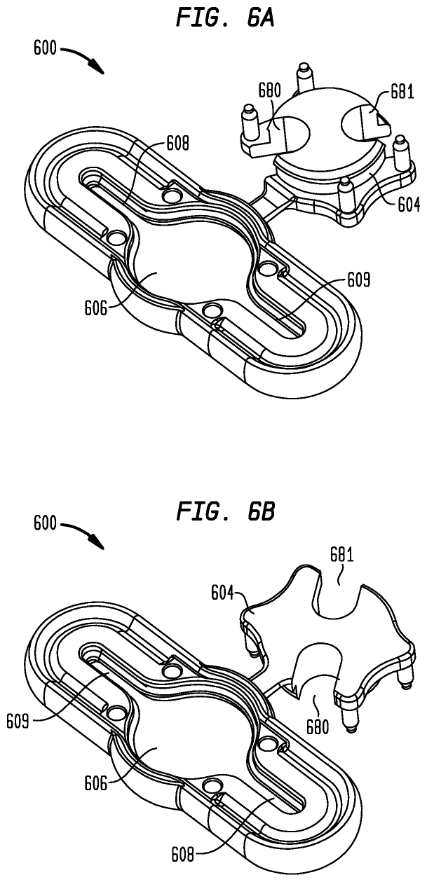

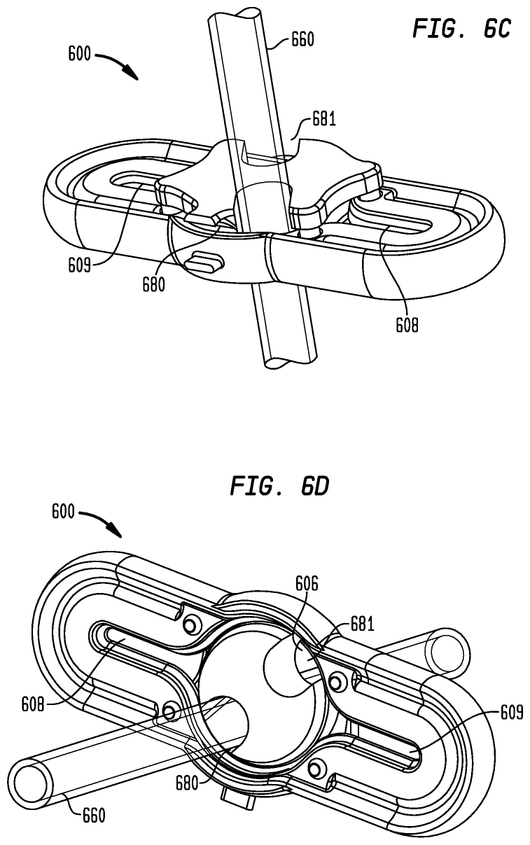

Referring to FIGS. 6A and 6B clamp 600 includes one or more pass-throughs 680, 681 in cap 604. Although two pass-throughs 680, 681 are shown in FIGS. 6A, 6B, 6C, 6D, other configurations are contemplated, such as one, three, four, five or more pass-throughs (not shown.) Pass-throughs 680, 681 do not align with slits 608, 609. Referring to FIGS. 6C and 6D, clamp 600 can be used with medical tubing 660. Pass-throughs 680, 681 are sized to substantially fit medical tubing 660 in size, that is sized to receive medical tubing 660 through e.g. passthrough 680 without clamping medical tubing 660 while not permitting substantial movement of medical tubing 660 within passthrough 680. Clamp 600 can be used when medical tubing 660 is IV tubing. Although one IV line 660 is shown in FIGS. 6C and 6D, clamp 600 can receive more than one IV line. Referring to FIG. 6D, clamp 600 allows IV lines to be individually controlled. For example, camp 600 allows unclamped medical tubing 660 to reside in opening 606 until clamped. Further, in various configurations (not shown), all the IV lines are gathered in the clamp, at least one IV line is clamped and at least one IV line is not clamped or all IV lines clamped or all IV lines are not clamped. This has the advantage of keeping the various IV lines from coming free and organizes the lines into one place where the user, for example a nurse, can easily manage multiple lines. The IV lines can be numbered or color coded to aid in organization as well. It will be understood that the size of a clamp can vary suitably with the number of IV lines to be accommodated. Referring again to FIGS. 1A, 1B, 1C, 1D, clamp 100 can be used with medical tubing, for example hemodialysis tubing or intravenous (IV) tubing or peripherally inserted central catheter (PICC) tubing. Suitable IV or PICC tubing can include a single tube. Alternately IV tubing can include two, three, four, or more tubes, either connected to a common insertion location to the patient or gathered together from different insertion locations. Suitable hemodialysis or PICC tubing can include two tubes, or alternately can include three tubes for example when in addition to attaching to two principle hemodialysis or PICC lumens the catheter includes a third lumen for non-principle-related access. When used with hemodialysis or PICC tubing with two or more tubes, one or two or more clamps 100 with one opening 106 can be used. Referring to FIG. 5, the communication between opening 506 and slits 508, 509 allows tubes 564, 574 to slide back and forth between opening 506 and slit 508 when clamp 500 is not locked by cap 504. Connector 562 includes tube 564, on/off clamp 566, and end piece 568. On/off clamp 566 and end piece 568 can be red in color (not shown).

Connector 572 includes tube 574, on/off clamp 576, and end piece 578. On/off clamp 576 and end piece 578 can be blue in color (not shown). Each of on/off clamps 566, 576 can be a Halkey Roberts clamp. When the tubes 564, 574 are disposed in slits 508, 509 tubes 564, 574 are clamped. When cap 504 is disposed in opening 506 (configuration not shown), cap 504 prevents tubes 564, 574 from sliding out. Referring again to FIG. 4, when cap 404 is disposed in the opening (not shown) of clamp 400, cap 404 prevents tubes 464, 474 from sliding out. When cap 404 is disposed in opening 406 clamp 400 is locked. Referring to FIG. 4 and FIG. 5, caps 404, 504 are capable to flex between an open position and a locked position. FIG. 5 shows an open position with engaging surface 510 disposed outside opening 506. FIG. 4 shows a locked position with the engaging surface (not shown) disposed within opening (not shown) thereby locking clamp 400. In operation, a method for clamping tubes 564, 574 can include passing the tube through opening 506, sliding tubes 564, 574 each aside towards slits 508, 509 respectively; and securing a cap 504 in opening 506 so as to lock the clamp 500, preventing the tubing from entering opening 506.

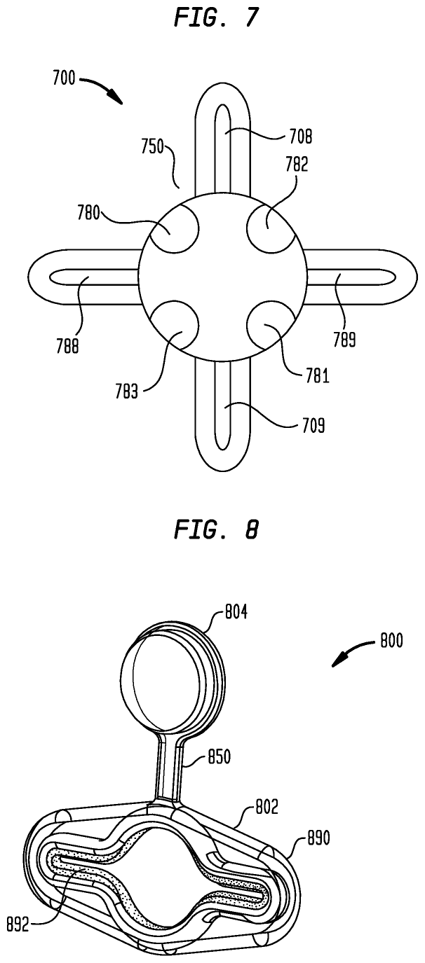

Referring to FIG. 7, it will be understood that clamp 700 can include more than two slits, that is at least three slits, for example three slits (not shown), four slits 708, 709, 790, 791, or more (not shown). Clamp 700 can include plate 702 and an opening (not shown) and slits 708, 709, 788, 789, wherein slits 708, 709, 788, 789 are arranged such that each is connected to opening 706. Clamp 700 includes hinge 750 and pass-throughs 780, 781, 782, 783. FIG. 7 shows clamp 700 with the opening as a common opening. Alternately, in the case of at least three slits, the present clamp can include a plate include a plurality of openings and the at least three slits, wherein each opening is connected to at least two of the at least three slits. Further, a method of clamping can include passing one or more tube through one, two, three, or more openings in a clamp with one, two, three, four, or more slits.

Referring to FIG. 8, it will be understood that clamp 800 can include plate 802 in two pieces, including sheath portion 890 and insert 892. Insert 892 aids plate 802 in holding its shape so that sheath 890 can contain a soft material optimized for hinge 850. Insert 892 can be imbedded into sheath 890, for example over molded. Sheath 890 can be integrally formed with hinge 850 and cap 804. Thus, plate 802, cap 804, and hinge 850 contain a first material and plate 802 includes insert 892 containing a second material. The first material can be softer than the second material. The first material is selected to sufficiently soft that hinge 850 flexes to open and close clamp 800 and to be sufficiently hard for cap 804 to hold its shape. The second material is selected to optimize plate 802 holding its shape, through the support of plate 802 by insert 892. The first and second materials can be different polymeric materials from each other. Suitable polymeric materials include thermoplastic, rubber, elastomer, and the like. For example, suitable materials include polypropylene, polyurethane, polyvinyl chloride, polyethylene coated polyvinyl chloride, silicone, and the like. Suitable materials for the first and second materials include conventional medical grade materials, for example medical grade polymeric materials. The second material can be metal. The different materials can be capable of being bonded. The different materials can both be polymeric. A suitable combination is polypropylene for the first material and polyurethane for the second material.

It will be understood that combinations of features other than those shown in FIGS. 1A, 1B, 1C, 1D, 2A, 2B, 2C, 2D, 3A, 3B, 3C, 3D, 4, 5, 6A, 6B, 6C, 6D, 7, 8 are contemplated. For example, while FIG. 4 shows clamp 400, a dual slit reinforced clamp as for clamp 200 shown in FIGS. 2A, 2B, 2C, and 2D, FIG. 5 shows clamp 500, a dual slit clamp that does not include a reinforcing outer edge as shown for clamp 100 having a single slit 108 shown in FIGS. 1A, 1B, 1C, 1D. Thus, for example a single slit clamp can optionally include a reinforcing edge and/or can include pairs of holes/prongs to be a single slit clamp not reinforced and not strengthened, a reinforced single slit clamp, a strengthened single slit clamp or a strengthened reinforced single slit clamp. Likewise, for example, a dual slit clamp can optionally include a reinforcing edge and/or can include pairs of holes/prongs to be a dual slit clamp not reinforced or strengthened, a reinforced dual slit clamp, a strengthened dual slit clamp or a strengthened reinforced dual slit clamp. Further, the above-described clamps can optionally include one or more pass-throughs. Further, it will be understood that the above-described clamps can include more than two slits, for example three slits, four slits, or more, with one, two, three, or more openings in a clamp with one, two, three, four, or more slits. Further, the above-described clamps can optionally include a plate with an insert supporting the plate.

It will be apparent to those skilled in the art that numerous modifications and variations of the described examples and embodiments are possible in light of the above teachings of the disclosure. The disclosed examples and embodiments are presented for purposes of illustration only. Other alternate embodiments can include some or all of the features disclosed herein. Therefore, it is the intent to cover all such modifications and alternate embodiments as can come within the true scope of this invention, which is to be given the full breadth thereof. Additionally, the disclosure of a range of values is a disclosure of every numerical value within that range, including the end points. Additionally, about a value denotes the value to within measurable limits or within ±10% if the measurable limits cannot be specified.

We claim:

1. A clamp comprising:
a plate comprising an opening and a slit in communication with the opening,
wherein the slit is sized to receive a tubing and prevent fluid passage through the tubing when the tubing is placed in the slit,
wherein the plate comprises a first bounding edge defining the opening and a second bounding edge defining the slit,
wherein the first and second bounding edges are continuous; and
a cap flexibly attached to the plate and comprising an engaging surface corresponding to the opening,
wherein the first bounding edge comprises a receiving surface corresponding to the engaging surface,
wherein the cap can be secured in the opening, preventing the tubing from entering the opening.

2. The clamp of claim 1, wherein the opening is adjacent the slit.

3. The clamp of claim 1, wherein the receiving surface has a profile that is flat or convex.

4. The clamp of claim 1, wherein the receiving surface has a shape and a size about the same as a corresponding shape and size of the engaging surface.

5. The clamp of claim 1, wherein the cap comprises a base portion at an end attached to the plate, a taper portion at an opposing end, and a plug portion between the base portion and the taper portion, wherein the plug portion comprises the engaging surface.

6. The clamp of claim 1, wherein the cap comprises a rib extending from the engaging surface.

7. The clamp of claim 6, wherein the rib is adjacent the optional taper portion of the cap.

8. The clamp of claim 1, comprising a hinge between the cap and the plate, wherein the hinge attaches the cap to the plate.

9. The clamp of claim 8, wherein the plate, cap, and hinge comprise a first polymeric material.

10. The clamp of claim 9, wherein the plate comprises an insert comprising a second polymeric material.

11. The clamp of claim 8, wherein the hinge comprises a first polymeric material and the plate and cap comprise a second polymeric material bonded to the first polymeric material.

12. The clamp of claim 1, wherein the plate comprises one or more holes through the plate in a region of the plate around the opening and where the cap comprises a matching one or more prongs.

13. The clamp of claim 1, wherein the clamp further comprises a reinforcing outer edge.

14. The clamp of claim 1, wherein the clamp further comprises at least one additional slit.

15. The clamp of claim 14, wherein the clamp further comprises at least another additional slit such that the clamp comprises at least three slits.

16. The clamp of claim 15, wherein the at least three slits are arranged such that each is connected to the opening.

17. The clamp of claim 14, wherein the plate comprises a one or more openings wherein each opening is connected to at least one of the at least two slits.

18. The clamp claim 1, wherein the cap comprises at least one non-clamping pass-through, wherein the non-clamping pass-through is not aligned with any slit which allows fluid passage through the tubing.

19. A method for clamping a tube comprising:
passing the tube through an opening in a plate of a clamp according to claim 1;
sliding the tube aside towards a slit in the clamp; and
securing a cap in the opening so as to lock the clamp, preventing the tubing from entering the opening, wherein the cap is attached to the plate.
